(19) United States
(12) Patent Application Publication
(10) Pub. No.: US 2008/0286588 A1
(43) Pub. Date: Nov. 20, 2008

(54) METALLIC COMPONENT WITH WEAR AND CORROSION RESISTANT COATINGS AND METHODS THEREFOR

(75) Inventors: Darren R. Burgess, Charlotte, NC (US); Mark S. Wabalas, Huntersville, NC (US); Glenn A. Rupp, Belmont, NC (US)

Correspondence Address: TREGO, HINES & LADENHEIM, PLLC, 9300 HARRIS CORNERS PARKWAY, SUITE 210, CHARLOTTE, NC, 28269-3797, US

(73) Assignee: BIOMEDFLEX, LLC, Huntersville, NC (US)

(21) Appl. No.: 11/750,590

(22) Filed: May 18, 2007

(51) Int. Cl. B32B 15/04; A61F 2/06; B32B 9/00; C23C 16/00

(52) U.S. Cl. 428/469; 427/249.1; 428/457; 623/1.46

(57) ABSTRACT

A component shielded with layers for impeding wear and corrosion includes: a metallic member having an outer surface, and a matrix of layers including a carbon-based layer and at least one oxide layer disposed on the outer surface. The layers may be formed by deposition or by other methods.

### BACKGROUND OF THE INVENTION

This invention relates generally to wear and corrosion resistant layers and more particularly to their application to metallic components.

Metals are often used to construct components placed in chemically and physically aggressive environments. For example, metallic components such as prosthetics, plates, screws and stents are often implanted into human or animal bodies. When placed in such conditions, metallic components are subject to a variety of corrosive chemicals and processes. One such process is electrochemical in nature and is known as galvanic corrosion. This process results in damage to the component often via the leaching of metal ions therefrom, which can be harmful to the body in which the component is placed.

Known types of coatings and treatments are used to protect metallic components from wear and corrosion. However, such coatings and treatments are not ultimately effective and due to their inadequacy may leave areas of a metallic component exposed. Such exposed areas are subject to a greater degree of wear and corrosion.

### BRIEF SUMMARY OF THE INVENTION

These and other shortcomings of the prior art are addressed by the present invention, which according to one aspect provides a component shielded with biocompatible layers for impeding wear and corrosion. The component includes: (a) a metallic member having an outer surface; (b) a first oxide layer disposed on the outer surface; and (c) a carbon-based layer disposed on the first oxide layer.

According to another aspect of the invention, a component shielded with layers for impeding wear and corrosion includes: (a) a metallic member having an outer surface; (b) a carbon-based layer disposed on the outer surface; and (c) an oxide layer disposed over the carbon-based layer.

According to another aspect of the invention, a method of producing a component shielded with layers for impeding wear and corrosion includes: (a) providing a metallic member having an outer surface; (b) depositing a carbon-based layer on the outer surface; and (c) forming an oxide layer over the carbon-based layer.

According to another aspect of the invention, a method of producing a component shielded with layers for impeding wear and corrosion includes: (a) providing a metallic member having an outer surface; (b) forming a first oxide layer on the outer surface; and (c) depositing a carbon-based layer on the first oxide layer.

### BRIEF DESCRIPTION OF THE DRAWINGS

The invention may be best understood by reference to the following description taken in conjunction with the accompanying drawing figures in which:

FIG. 1 is a schematic side view of a stent treated in accordance with the present invention;

FIG. 2 is a cross-sectional view of a portion of a near surface region of the stent of FIG. 1 with a first oxide layer and a carbon-based layer thereon;

FIG. 3 is a cross-sectional view of a portion of the stent of FIG. 2 after thickening the first oxide layer through formation or disposition of a new oxide layer;

FIG. 4 is a cross-sectional view of a portion of the stent of FIG. 2 after having a second oxide layer deposited thereon; and

FIG. 5 is a cross-sectional view of a portion of a near surface region of a metallic component having a carbon-based layer and an oxide layer thereon.

### DETAILED DESCRIPTION OF THE INVENTION

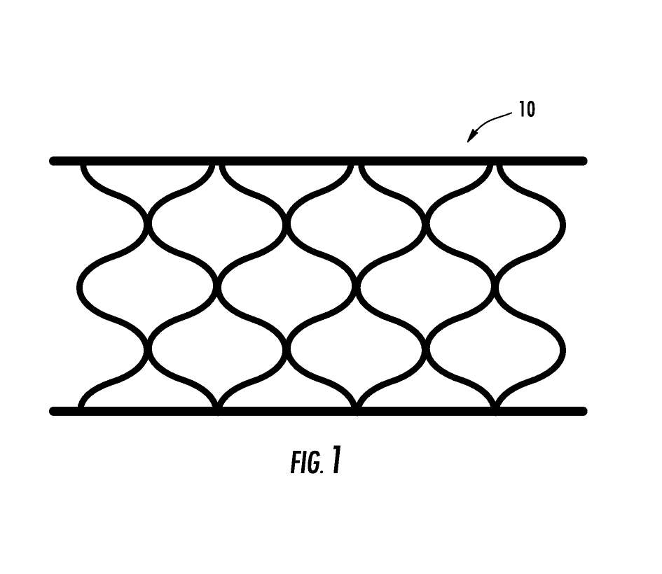

Referring to the drawings wherein identical reference numerals denote the same elements throughout the various views, FIG. 1 depicts an exemplary stent 10 constructed in accordance with the present invention. The stent 10 has a lattice-like construction of slender, elongated members. In the expanded condition, the stent 10 is generally cylindrical. It should be noted that the stent 10 is merely used as an example, and the matrix of layers and method of the present invention is useful for any metallic component which is exposed to corrosion or wear. Non-limiting examples of components that may be coated as described herein include medical instruments, stents, implanted devices, orthopedic implants, plates, screws, prosthetics and dental and orthodontic appliances and implants. The stent 10 is made from a metal, metallic compound, or metal alloy. One example of a suitable material often used for implants and stents is an alloy of nickel and titanium generally referred to as NITINOL. Other known metals used for implants and medical components include titanium, stainless steels, cobalt chrome, cobalt-chromium-molybdenum, trabecular, titanium-aluminum-niobium and similar materials. The coatings and methods of the present invention are also useful with other substrate materials, non-limiting examples of which include nickel-based superalloys such as RENE 80, INCONEL alloys, WASPALLOY, and HASTELLOY, stainless steels, Invar iron-nickel alloy, Kovar nickel-cobalt alloy, and cobalt chrome.

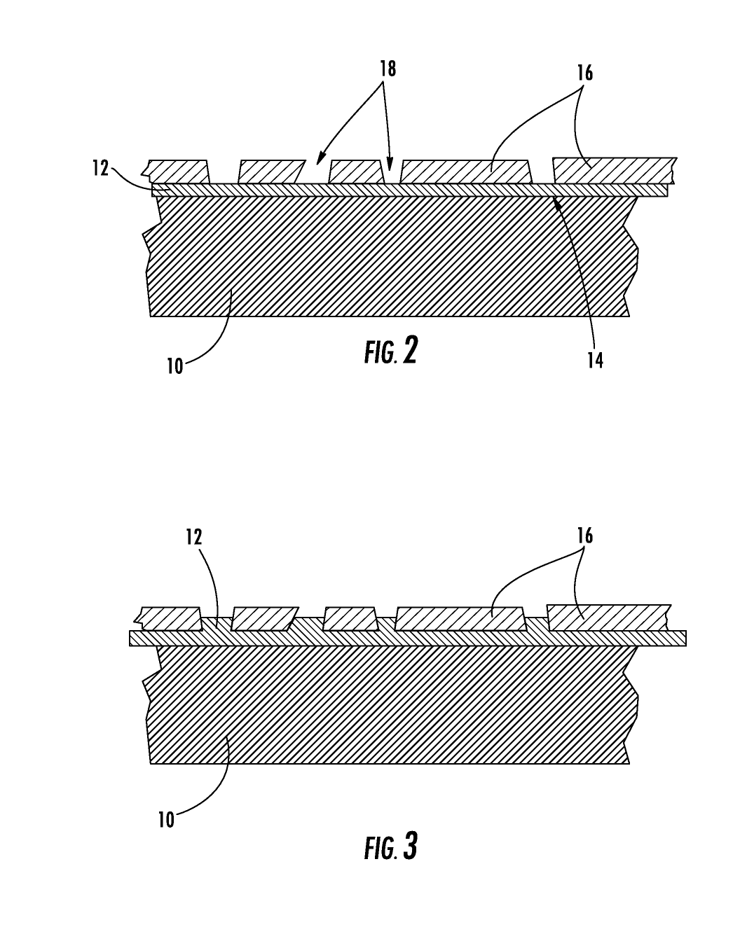

The stent 10 has at least two layers thereon which shield it against wear and corrosion. In the example shown in FIG. 2, the stent 10 has an oxide layer 12 disposed on its outer surface 14 and a carbon-based layer 16 disposed on the oxide layer 12. It will be understood that the term “outer surface” is used herein to mean generally any surface of a substrate which is exposed to the surrounding environment and does not necessarily imply any specific geometrical feature of the substrate.

The oxide layer 12 impedes corrosion of the underlying substrate. Any element contained in the stent 10 and present at the outer surface 14 or any element that can be delivered to the outer surface 14, such as by chemical vapor transport, which forms a solid-phase compound with oxygen at the processing temperature, may be used. Non-limiting examples of elements which meet the above criteria include Al, As, Ba, Bi, Ca, Co, Cr, Fe, Ga, Ge, Hf, Mo, Mn, Nb, Pb, Rh, Ru, Sc, Si, Sn, Sr, La, Ni, Cu, Ta, Ti, V, W, Y, Zn, and Zr. The oxide layer 12 may be an oxide of a primary constituent metal of the stent 10 or an oxide of another material.

The carbon-based layer 16 is a biologically inert or biocompatible material. The carbon-based layer 16 resists wear and acts as a barrier against biofluids, chemicals, moisture, etc. The carbon-based layer 16 exhibits very low surface roughness, which reduces wear and damage to surfaces (e.g. artery walls) in contact with the stent 10, causes less buildup and adhesion of other materials, and facilitates extraction because it does not tend to adhere to other materials. The applied benign surface treatment assists in limiting the likelihood of blood clot formation.

One example of a suitable material for the carbon-based layer is referred to as a “diamond-like carbon” and is essentially pure carbon, has a non-crystalline microstructure, and exhibits a flexural capability with a strain rate of approximately 8% or better. The structure and bonding of the carbon layer enable it to endure significant vibration and deformation without cracking or detaching from the substrate or delaminating. Carbon-based layers with such properties may be applied by a plasma assisted chemical vapor deposition (CVD) process and may be obtained from BioMedFlex LLC, Huntersville, N.C., 28078.

Another example of a known material suitable for the carbon-based layer REF is a so-called “diamond-like nanocomposite” comprising a diamond-like carbon network stabilized by hydrogen, and a glass-like silicon network stabilized by silicon, with both networks mutually stabilizing each other. An example of such a material is described in U.S. Pat. No. 5,352,493 to Dorfman et al.

The carbon-based layer 16 is superior to other coatings used for similar purposes, but is not perfect. Even with careful application, the carbon-based layer 16 may contain flaws. FIG. 2 illustrates a portion of the stent 10 with the carbon-based layer 16. Several voids 18 (i.e. cracks or pockets) are shown. It is noted that the relative size of the voids 18 are shown greatly exaggerated for illustrative purposes. If not for the presence of the oxide layer 12 under the carbon-based layer 16, the voids 18 would expose the outer surface 12 of the stent 10. If used in an environment such as a human or animal body without further treatment, these exposed surface portions would be more vulnerable to corrosion and wear processes. Over time, these degrading processes could cause undesirable changes in the structure of the stent 10, or could leach materials from the stent 10 into the body with possibly harmful effects. The presence of the oxide layer 12 underneath the carbon-based layer 16 prevents or greatly reduces corrosion and wear.

Various methods may be used to form the oxide layer 12. One method is to form an oxide layer including one or more metals found at or near the outer surface 14 by exposing the outer surface 14 to an aqueous acid solution, e.g. aqueous solutions of nitric acid, hydrofluoric acid, sulfuric acid, or hydrochloric acid. An exemplary acid treatment process is as follows. First, the stent 10 is completely immersed in an aqueous solution of nitric acid. A suitable acid solution should be least ten volumetric percent water and at a maximum temperature of 80° C. (176° F.). The stent 10 is maintained in the acid solution for at least 2 minutes. The stent 10 is then removed from the acid solution and rinsed with distilled water to remove any traces of the acid.

Another method is to form an oxide layer including one or more metals found at or near the outer surface 14 by exposing the outer surface 14 to oxygen-containing plasma, e.g. plasmas of O₂, CO₂, or O₃. An example of this process is as follows. First, the stent 10 is placed in a vacuum chamber (not shown) having a base pressure nominally 1×10⁻³ Pascal (1×10⁻⁵ Torr) or lower. Next, ozone gas (O₃) is flowed into the vacuum chamber at a rate which is determined by a ratio of chamber volume to volumetric flow rate. The ratio should be 800 minutes or less. While the gas is flowing, a radio frequency (RF) plasma is struck with a generator of a known type operating at about 32.56 MHz coupled with an automatic impedance matching network to the vacuum chamber via a conductive feedthrough. The stent 10 is subjected to the plasma for approximately ten minutes.

Yet another method is to deposit an oxide layer onto the outer surface 14. An example of this method is as follows. The stent 10 is placed in a vacuum chamber having a base pressure nominally 1×10⁻³ Pascal (1×10⁻⁵ Torr) or lower. Optionally, gaseous argon is flowed into the vacuum chamber at a rate which is determined by a ratio of chamber volume to volumetric flow rate. The ratio should be 800 minutes or less. A gas phase precursor, the vapor of a liquid phase precursor or the vapor of an organic solution of a solid phase precursor is flowed into the vacuum chamber. The ratio of chamber volume to volumetric flow rate should be about 800 minutes or less. The precursor molecule should contain oxygen molecules. For example, a bismuth oxide layer may be deposited from tris(2,2,6,6-tetramethylheptane-3,5-dionato)bismuth. An RF plasma is struck with a generator of a known type operating at about 32.56 MHz coupled with an automatic impedance matching network to the chamber via a conductive feedthrough.

Any of the above methods may be used to create additional oxide layers. Depending on a variety of factors, such as the material comprising the stent 10 and its intended location in the body, any of these three methods may be used to re-create or thicken oxide layer 12 after the carbon-based layer 16 is applied. FIG. 3 illustrates the oxide layer 12 after thickening in this manner.

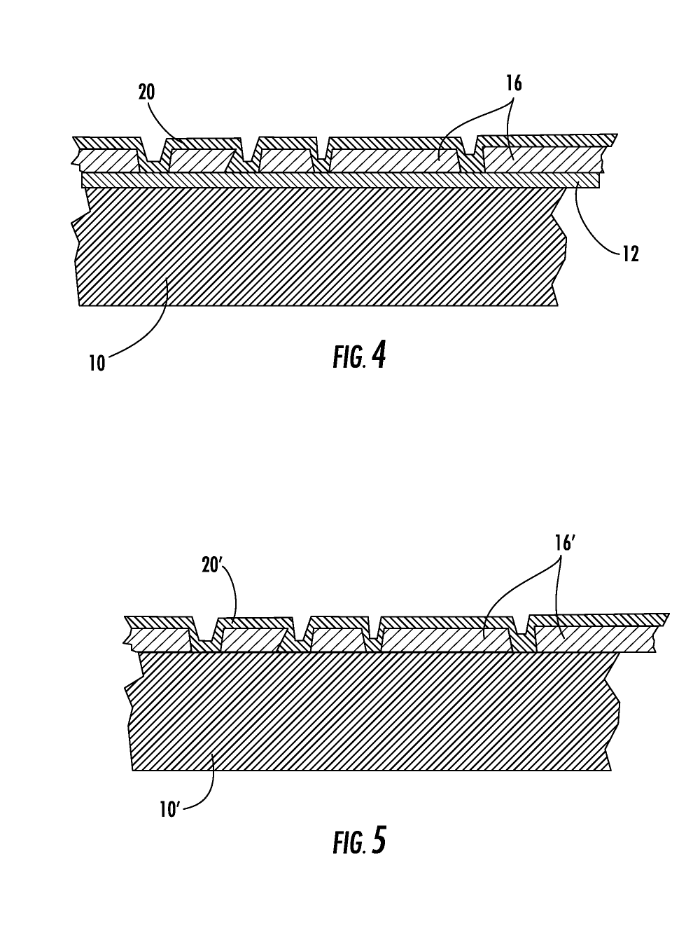

A oxide layer 20 (see FIG. 4) having a composition the same or different from that of the oxide layer 12 may be deposited after the carbon-based layer 16 via chemical vapor deposition as described above.

If desired, the stent 10 may be mechanically or electrochemically stressed as described above after formation of the layers 14, 16, and 20. Stressing the stent 10 will cause any weak areas in the layers to be exposed and reveal additional voids. An oxide layer formation process may be applied yet a third time to fill the new voids.

FIG. 5 shows a portion of a stent 10′ similar in construction to the stent 10 with an alternative arrangement of shielding layers thereon. The stent 10′ has a carbon-based layer 16′ disposed on its outer surface and an oxide layer 12′ disposed on the carbon-based layer 16′. The composition and application of these layers is the same as that of the oxide and carbon-based layers 20 and 16 described above. If desired, the stent 10 may be mechanically or electrochemically stressed, or both, before formation of the oxide layer 20′. This step will cause any weak areas in the carbon-based layer 16′ to be exposed and reveal additional voids.

It is known to apply anti-inflammatory or antibiotic coatings to the stent 10 to create so-called “drug-eluting” stents. While these coatings are medically effective, they also have a tendency to dissolve, thus exposing the base material of the stent 10. In contrast to the prior art, the stent 10 with the shielding layer arrangement described above will remain protected even when the drug coatings (if the two are combined) wear away. The resilient hard carbon layer also can stand alone as the sole anti-inflammatory surface treatment on a stent.

The foregoing has described a shielded component and a method for applying those layers. While specific embodiments of the present invention have been described, it will be apparent to those skilled in the art that various modifications thereto can be made without departing from the spirit and scope of the invention. Accordingly, the foregoing description of the preferred embodiments of the invention and the best mode for practicing the invention are provided for the purpose of illustration only and not for the purpose of limitation.

What is claimed is:

1. A component shielded with layers for impeding wear and corrosion comprising:
(a) a metallic member having an outer surface;
(b) a first oxide layer disposed on the outer surface; and
(c) a carbon-based layer disposed on the first oxide layer.

2. The shielded component of claim 1 wherein the first oxide layer comprises an oxide of a primary constituent metal of the metallic member.

3. The shielded component of claim 1 wherein the first oxide layer comprises an oxide of an element other than a primary constituent metal of the metallic member.

4. The component of claim 1 further comprising a second oxide layer comprising a stable oxide disposed over the carbon-based layer.

5. The component of claim 4 wherein:
(a) the carbon-based layer has at least one void therein which exposes a portion of the first oxide layer or the metallic member; and
(b) at least a portion of the second oxide layer is formed on the exposed portion of the first oxide layer or the metallic member.

6. The component of claim 4 wherein the second oxide layer comprises an oxide of a primary constituent metal of the metallic member.

7. The component of claim 4 wherein the second oxide layer comprises an oxide of an element other than that of a primary constituent metal of the metallic member.

8. The component of claim 1 wherein the carbon-based coating consists essentially of carbon in a non-crystalline microstructure.

9. The component of claim 1 in which the metallic member is a stent having a lattice structure.

10. The component of claim 1 wherein the metallic member comprises an alloy of Ni and Ti.

11. A component shielded with layers for impeding wear and corrosion comprising:
(a) a metallic member having an outer surface;
(b) a carbon-based layer disposed on the outer surface; and
(c) an oxide layer disposed over the carbon-based layer.

12. The component of claim 11 wherein:
(a) the carbon-based layer has at least one void therein which exposes a portion of the outer surface; and
(b) at least a portion of the oxide layer is formed on the exposed portion of the outer surface.

13. The component of claim 11 wherein the oxide layer comprises an oxide of a primary constituent metal of the metallic member.

14. The component of claim 11 wherein the oxide layer comprises an oxide of an element other than a primary constituent metal of the metallic member.

15. The component of claim 11 wherein the carbonaceous coating consists essentially of carbon in a non-crystalline microstructure.

16. The component of claim 11 wherein the metallic member comprises an alloy of Ni and Ti.

17. The component of claim 1 wherein the metallic member comprises Ti or an alloy thereof.

18. The component of claim 1 in which the metallic member is a stent having a lattice structure.

19. A method of producing a component shielded with layers for impeding wear and corrosion, comprising:
(a) providing a metallic member having an outer surface;
(b) depositing a carbon-based layer on the outer surface; and
(c) forming an oxide layer over the carbon-based layer.

20. The method of claim 19 wherein the carbon-based layer has at least one void therein which exposes a portion of the outer surface; and wherein the oxide layer is formed by contacting the metallic member with an aqueous acid solution, so as to cause in-situ oxide formation on the exposed portions of the outer surface.

21. The method of claim 19 wherein the carbon-based layer has at least one void therein which exposes a portion of the outer surface, and wherein the oxide layer is formed by:
(a) placing the metallic member in a chamber maintained at a vacuum;
(b) introducing oxygen molecules into the chamber; and
(c) providing adequate energy to the oxygen molecules and the metallic member so as to cause in-situ oxide formation on the exposed portions of the outer surface.

22. The method of claim 21 wherein step (c) is carried out by striking an RF plasma in the chamber.

23. The method of claim 19 wherein the oxide layer is formed by:
(a) placing the metallic member in a chamber maintained at a vacuum;
(b) introducing oxygen and an oxide precursor including molecules of at least one element other than oxygen into the chamber; and
(c) providing adequate energy to the oxygen molecules and the oxide precursor so as to cause oxide deposition over the carbon-based layer.

24. The method of claim 23 wherein step (c) is carried out by striking an RF plasma in the chamber.

25. The method of claim 19 in which the carbon-based layer consists essentially of carbon in a non-crystalline microstructure.

26. The method of claim 19 wherein the metallic member comprises an alloy of Ni and Ti.

27. The method of claim 19 wherein the oxide layer comprises an oxide of a primary constituent metal of the metallic member.

28. The method of claim 19 wherein the oxide layer comprises an oxide of an element other than a primary constituent metal of the metallic member.

29. A method of producing a component shielded with layers for impeding wear and corrosion, comprising:
(a) providing a metallic member having an outer surface;
(b) forming a first oxide layer on the outer surface; and
(c) depositing a carbon-based layer on the first oxide layer.

30. The method of claim 29 wherein the first oxide layer is formed by contacting the metallic member with an aqueous acid solution, so as to cause in-situ oxide formation on the outer surface.

31. The method of claim 29 wherein the first oxide layer is formed by:
(a) placing the metallic member in a chamber maintained at a vacuum;
(b) introducing oxygen molecules into the chamber; and
(c) providing adequate energy to the oxygen molecules and the metallic member so as to cause in-situ oxide formation on the outer surface.

32. The method of claim 31 wherein step (c) is carried out by striking an RF plasma in the chamber.

33. The method of claim 29 wherein the first oxide layer is formed by:
(a) placing the metallic member in a chamber maintained at a vacuum;
(b) introducing oxygen molecules and an oxide precursor comprising molecules of at least one element other than oxygen into the chamber; and
(c) providing adequate energy to the oxygen molecules and the oxide precursor so as to cause oxide deposition on the carbon-based layer.

34. The method of claim 33 wherein step (c) is carried out by striking an RF plasma in the chamber.

35. The method of claim 29 wherein the first oxide layer comprises an oxide of a primary constituent metal of the metallic member.

36. The method of claim 29 wherein the first oxide layer comprises an oxide of an element other than a primary constituent metal of the metallic member.

37. The method of claim 29 further comprising forming a second oxide layer of a stable oxide over the carbon-based layer.

38. The method of claim 37 wherein the second oxide layer is formed by:
(a) placing the metallic member in a chamber maintained at a vacuum;
(b) introducing oxygen molecules into the chamber;
(c) introducing molecules of at least one element other than oxygen into the chamber; and
(d) providing adequate energy to the oxygen molecules and the oxide precursor so as to cause oxide deposition over the carbon-based layer.

39. The method of claim 38 wherein step (d) is carried out by striking an RF plasma in the chamber.

40. The method of claim 37 wherein the carbon-based layer has at least one void therein which exposes a portion of the first oxide layer or the metallic member, and wherein the second oxide layer is formed by contacting the metallic member in an aqueous acid solution, so as to cause in-situ oxide formation on the exposed portions of the first oxide layer or the metallic member.

41. The method of claim 37 wherein the carbon-based layer has at least one void therein which exposes a portion of the first oxide layer or the metallic member, and wherein the second oxide layer is formed by:
(a) placing the metallic member in a chamber maintained at a vacuum;
(b) introducing oxygen molecules into the chamber; and
(c) providing adequate energy to the oxygen molecules and the oxide precursor so as to cause in-situ oxide formation deposition on the exposed portions of the first oxide layer.

42. The method of claim 41 in which step (c) is carried out by striking an RF plasma in the chamber.

43. The method of claim 37 wherein the second oxide layer comprises an oxide of a primary constituent metal of the metallic member.

43. The method of claim 37 wherein the second oxide layer comprises an oxide of an element other than a primary constituent metal of the metallic member.
